(19) United States
(12) Patent Application Publication
(10) Pub. No.: US 2026/0207625 A1
(43) Pub. Date: Jul. 23, 2026

(54) New medical use of 3a-ethynyl-3ß hydroxyandrostan-17-one oxime

(71) Applicant: Umecrine Cognition AB, Solna (SE)

(72) Inventors: Thomas P. Blackburn, Benfleet (GB); Magnus Doverskog, Stockholm (SE); Lars Öhman, Stockholm (SE)

(21) Appl. No.: 19/140,401

(22) PCT Filed: Dec. 21, 2023
(86) PCT No.: PCT/EP2023/087503

§ 371 Date: Jun. 18, 2025

(30) Foreign Application Priority Data
Dec. 22, 2022 (EP) 22216200.0

(51) Int. Cl. A61K 31/568; A61P 25/16

(52) U.S. Cl. CPC A61K 31/568; A61P 25/16

(57) ABSTRACT

The present invention is directed to the compound golexanolone for use in the treatment of Parkinson's Disease (PD) or for use in the treatment of L-dopa Induced Dyskinesia (LID) in Parkinson's Disease (PD) patients. Further, the present invention is directed to the compound golexanolone or a pharmaceutically acceptable salt thereof, for use in the treatment of Parkinson's Diseases (PD) patients, in particular PD patients exhibiting a L-dopa Induced Dyskinesia (LID).

### FIELD OF THE INVENTION

The present invention is directed to the compound golexanolone for use in the treatment of Parkinson's Disease (PD) or for use in the treatment of L-dopa Induced Dyskinesia (LID) in Parkinson's Disease (PD) patients. Further, the present invention is directed to the compound golexanolone for use in the treatment of Parkinson's Disease (PD) patients, in particular PD patients exhibiting a L-dopa Induced Dyskinesia (LID).

### BACKGROUND OF THE INVENTION

Parkinson's Disease (PD) is a degenerative condition of the brain associated with motor symptoms (slow movement, tremor, rigidity, walking, and imbalance) and a wide variety of non-motor complications (fatigue, cognitive impairment, mental health disorders such as depression and anxiety, sleep disorders and pain and other sensory disturbances) (Lewitt, A P. and Chaudhuri, K R. 2020. Parkinsonism and Related Disorders, 80: 57-512). Motor impairments, such as dyskinesias (involuntary movements) and dystonias (painful involuntary muscle contractions) contribute to limitations in speech, mobility, and restrictions in many life areas. Progression of these symptoms results in high rates of disability and care requirements. Many people with PD also develop dementia during the course of their disease (WHO 13 Jun. 2022: Parkinson disease (who.int).

While PD is the most common movement disorder, other movement disorders such as atypical parkinsonism exist such as multiple system atrophy (MSA), progressive supranuclear palsy (PSP), corticobasal degeneration (CBD), and dementia with Lewy bodies (DLB) (Przewodowska, D. et al. 2021. Frontiers in Molecular Neuroscience., August 2021, Volume 14, Article 720220). Some movement disorders have similar symptoms to PD such as tremor, slow movement, and rigidity. All movement disorders share the same challenges as PD regarding diagnostic and treatment gaps and access to medication, particularly in low- and middle-income countries (LMIC) (WHO 13 June 2022: Parkinson disease (who.int).

Risk factors for PD include increasing age, although younger people can be affected as well. Men are more affected than women. The cause for PD is not known but is thought to arise from a complex interaction between genetic factors and exposure to environmental factors such as pesticides, solvents, and air pollution throughout life (WHO 13 Jun. 2022: Parkinson disease (who.int). Levo-dihydroxyphenylalanine (L-DOPA) is the most effective treatment for Parkinson's disease; however, most patients develop uncontrollable abnormal involuntary movements known as L-DOPA-induced dyskinesia (Nishijima H et al: Neurobiology of Disease 143 (2020); pp. 1-13; 104979).

Levodopa-induced dyskinesia (LID) is a form of dyskinesia associated with levodopa (L-DOPA), used to treat Parkinson's disease. It often involves hyperkinetic movements, including chorea, dystonia, and athetosis (Gerlach M et al: December 2011; Journal of Neural Transmission. 118 (12): pp. 1659-1660).

In the context of Parkinson's disease (PD), dyskinesia is often the result of long-term dopamine therapy. These motor fluctuations occur in up to 80% of PD patients after 5-10 years of L-DOPA treatment (Ahlskog J E et al 2001: Mov Disord. 16 (3): pp. 448-458), with the percentage of affected patients increasing over time (Obeso J A; et al. 2000; Neurology. 55 (S4): 513-520).

Based on the relationship with levodopa dosing, dyskinesia most commonly occurs at the time of peak L-DOPA plasma concentrations and is thus referred to as peak-dose dyskinesia (PDD). As patients advance, they may present with symptoms of diphasic dyskinesia (DD), which occurs when the drug concentration rises or falls, and “OFF” period dystonia which occurs when the exposure of L-DOPA becomes low leading to prolonged spasms and postures (Fabbrini, A. and Guerra, A. 2021. Journal of Experimental Pharmacology, 13: 469-485). If dyskinesia becomes too severe or impairs the patient's quality of life, a reduction in L-Dopa might be necessary, however, this may be accompanied by a worsening of motor performance and “OFF” period dystonia. Therefore, once established, LID is difficult to treat (Thanvi B et al 2007: Postgraduate Medical Journal. 83 (980): pp. 384-388).

In 2017, the FDA approved amantadine (Gocovri®, Adamas Pharmaceuticals) as first-line treatment for levodopa-induced dyskinesia (LID) in Parkinson's patients.

Parkinson's disease (PD) is a neurodegenerative disease that involves multiple neuronal systems—a fact readily apparent given the broad spectrum of motor and non-motor symptoms that patients with this condition develop over time (Terkelsen, M H. et al., 2022. Current Neurology and Neuroscience Reports., doi.org/10.1007/s11910-022-01245-z). However, the clinical diagnosis of PD is based on the classic motor symptoms bradykinesia, rigidity and resting tremor, caused by loss of dopaminergic projections from the substantia nigra pars compacta (SNc) to the striatum, the primary input nucleus of the motor corticostriatal circuitry. Parkinsonian symptoms are also a major feature of atypical parkinsonism, a rather heterogeneous group of neurodegenerative diseases, that can, in the beginning, be misdiagnosed as PD. Within the basal ganglia, the dopaminergic neurons from the SN pars compacta (SNc) exert modulation on the direct and indirect pathways, the two neural circuits connecting the striatum to the thalamus, decreasing the overall inhibitory output and thereby refining its processing. The inhibitory projection neurons of both these pathways, the projection medium spiny neurons and the efferents of globus pallidus, use γ-aminobutyric acid (GABA) as a transmitter. In fact, one-third of all brain synapses use GABA for inhibition.

Several reports support that enhanced GABAergic neurotransmission contribute to the pathogenesis of Parkinson's disease, and to associated motor symptoms. In particular, the report from Heo et al (Curr Biol. 2020 Jan. 20; 30(2): pp. 276-291) shows that in animal models of Parkinson's disease the levels of GABA are increased in substantia nigra pars compacta, especially in activated astrocytes. These increased levels of GABA enhance activation of GABA_{A} receptors leading to reduced expression of tyrosine hydroxylase in neurons which, in turn, is responsible for the motor deficits in these animal models.

This effect occurs in neurons which have not yet died, and it is therefore possible to act on these mechanisms to improve motor deficits and quality of life of the patients. Heo et al (Curr Biol. 2020 Jan. 20; 30(2): pp. 276-291) also show that inhibiting MAOb (monoamineoxidase b) reduces GABA levels and restores the expression of tyrosine hydroxylase in neurons, which is associated with improvement of motor deficits. Tyrosine hydroxylase expression is also restored by blocking GABA_{A} receptors containing the α5 subunit, supporting that enhanced activation of GABA_{A} receptors mediates the inhibition of tyrosine hydroxylase expression and motor deficits.

Other reports support that the alterations in substantia nigra pars compacta are transmitted to other brain areas, leading to alterations in cerebellum (Wu et al. Brain. 2013 March; 136 (Pt 3): pp. 696-709); Rusholt et al, Brain Pathol. 2020 May; 30(3): pp. 576-588), including reduced levels of tyrosine hydroxylase (Hurley et al. Eur J Neurosci. 2003 November; 18(9): pp. 2668-2672) and appearance of alpha-synuclein aggregates (Seidel et al; Ann Neurol. 2017 June; 81(6): pp. 898-903) which also contribute to the anxiety (Wang et al, Transl Neurosci. 2021 Oct. 29; 12(1): pp. 415-424) and motor (Ballanger et al, J Neurol Neurosurg Psychiatry. 2008 October; 79(10): pp. 1110-1116); (Lewis et al, Can J Neurol Sci. 2013 May; 40(3): pp. 299-306); (Seidel et al, Ann Neurol. 2017 June; 81(6): pp. 898-903) and cognitive deficits in patients and animal models of Parkinson's disease.

In addition to the motor impairment, patients and animal models with Parkinson's disease also show cognitive impairment (Lindgren et al Eur J Neurosci. 2012 June; 35(12): pp. 1894-1907; (Leao et al, 2021 Behav Brain Res. 2021 Jul. 23; 410: 113349); (Schneider et al; Exp Neurol. 2021 January; 335:113514); and (Tian et al; Oxid Med Cell Longev. 2022 Jan. 4; 2022: 2792348).

The hallmark of any neurodegenerative disease is selective neuronal loss, which is associated with the activation of microglia and astrocytes (Dickson, D. W. Cold Spring Harb. Perspect. Med., 2(8), a009258, 2012). Neuroinflammation, with activation of microglia and astrocytes, plays a key role in the aetiology of Parkinson's Disease and other α-synucleinopathies (Kam T. I., et al; Neurobiol Dis. 2020 October; 144:105028); (Stefanova N; J. Parkinsons Dis., 12(s1), S105-S112; 2022); and (Chen K et al. Brain Sci., 13(4), 634; 2023). Neuroinflammation is linked to the accumulation and aggregation of α-synuclein (alphaSyn), the primary pathological feature of Parkinson's Disease.

The motor symptoms of Parkinson's disease are attributed to dopamine depletion, caused by neurodegeneration and extensive loss of dopaminergic neurons in substantia nigra, resulting in the loss of tyrosine hydroxylase (TH), the main dopamine-producing enzyme (Zhou, Z. D. et ai 2022; Cell. Mol. Life Sci., 79(12), 599). The gold standard marker in the identification of dopaminergic neurons is tyrosine hydroxylase (TH) and reduction of tyrosine hydroxylase (TH) is a measure of loss of dopaminergic neurons (Nagatsu T et al. J Neural Transm. 2019 April; 126(4): pp. 397-409).

Further, the pathogenesis of LID is complex, and different neurotransmitters such as dopamine, glutamine, adenosine, and gamma-aminobutyric acid play important role altering the normal physiology of direct and indirect pathway of cortico-basal ganglia-thalamic loop responsible for fine motor control (Pandey and Srivanitchapoom. 2017. Ann Indian Acad Neurol. 20: pp. 190-198). Several reports support that enhanced GABAergic neurotransmission contributes to the pathogenesis of L-DOPA induced dyskinesia in Parkinson's disease, and to associated motor symptoms. In particular, the report from Nishijima et al. (Nishijima H et al: Neurobiology of Disease 143 (2020); pp. 1-13; 104979) showing that in animal models of Parkinson's disease the levels of GABA are increased in the medial globus pallidus, corresponding to globus pallidus interna in humans.

Nishijima et al. (Nishijima H et al: Neurobiology of Disease 143 (2020); pp. 1-13; 104979) also show that inhibiting the GABAA receptors with the experimental GABAA receptor antagonist bicuculline alleviated LID in Parkinson's disease model rats pre-treated with L-DOPA.

In summary, in accordance with the classical network model of basal ganglia, degeneration of the neurons in the SNc (substancia nigra pars compacta) leads to an imbalance of GABA and glutamate neurotransmission in the nigrostriatal system in Parkinson's disease which is associated with motor deficits and other behavioral symptoms in Parkinson's disease. This is in contrast to, for example, liver disorders such as liver cirrhosis in which motor incoordination is caused by increased levels of ammonia in the blood and inflammation leading to the impaired function of the cerebellum as described by Balzano et al (Biomedicines. 2021 Aug. 12; 9(8):1002. doi: 10.3390/biomedicines9081002.).

The compound golexanolone, having the chemical name 3α-ethynyl-3β-hydroxyandrostan-17-one oxime, is a compound currently in clinical Phase II for the treatment of Hepatic Encephalopathy (HE). This compound is disclosed in WO 2008/063128 for use in various CNS disorders. WO 2015/114308 discloses the use of the compound 3α-ethynyl-3β-hydroxyandrostan-17-one oxime for the treatment of Hepatic Encephalopathy (HE). US patent application published as US2017/0348323, discloses a method for the treatment of hypersomnolence by administering the compound 3α-ethynyl-3β-hydroxyandrostan-17-one oxime. WO 2019/102040 discloses a pharmaceutical formulation of the compound golexanolone. WO 2022/223526 discloses the compound 3α-ethynyl-3β-hydroxyandrostan-17-one oxime for use in chronic liver diseases and symptoms related thereto.

### DESCRIPTION OF THE INVENTION

An aspect of the present invention is the compound golexanolone (3α-ethynyl-3β hydroxyandrostan-17-one oxime) of formula (I)

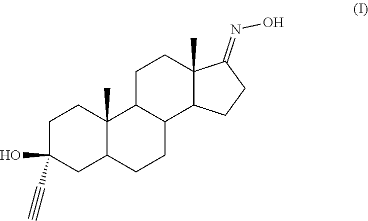 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of Parkinson's Disease (PD).

In another aspect of the present invention, the treatment is in Parkinson's Disease (PD) patients that exhibit a L-dopa Induced Dyskinesia (LID).

A further aspect of the present invention is the compound golexanolone (3α-ethynyl-3p hydroxyandrostan-17-one oxime) of formula (I)

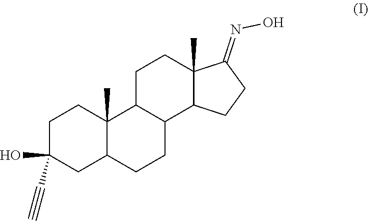 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of Parkinson's Disease (PD) patients.

In another aspect of the present invention, the PD patients to be treated exhibit a L-dopa induced dyskinesia (LID).

A further aspect of the present invention is the compound golexanolone (3α-ethynyl-3β hydroxyandrostan-17-one oxime) of formula (I)

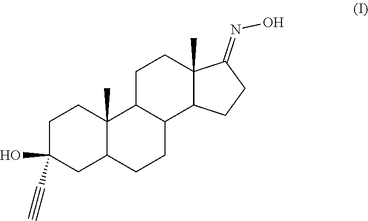 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of L-dopa induced dyskinesia (LID) in PD patients.

In particular, the present invention relates to the following embodiments:
i) The compound golexanolone (3α-ethynyl-3β hydroxyandrostan-17-one oxime) of formula (I)

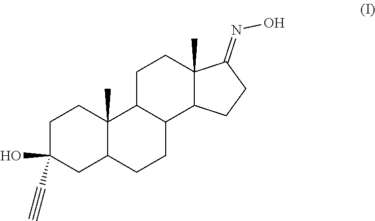 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of Parkinson's Disease (PD).
ii) The compound golexanolone for use according to i), wherein the treatment is in Parkinson's Disease (PD) patients that exhibit a L-dopa Induced Dyskinesia (LID).
iii) The compound golexanolone (3α-ethynyl-3β hydroxyandrostan-17-one oxime) of formula (I)

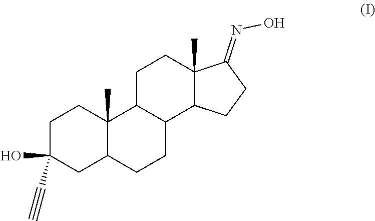 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of Parkinson's Disease (PD) patients.
iv) The compound golexanolone for use according to iii), wherein the Parkinson's Disease (PD) patients exhibit a L-dopa Induced Dyskinesia (LID).
v) The compound golexanolone (3α-ethynyl-3β hydroxyandrostan-17-one oxime) of formula (I)

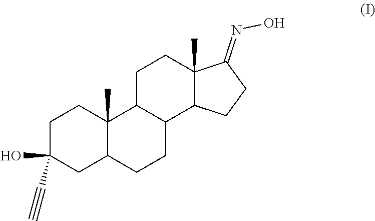 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of L-dopa Induced Dyskinesia (LID) in Parkinson's Disease (PD) patients.
vi) The compound golexanolone for use according to any one of i) to v), wherein the Parkinson's Disease (PD) comprises motor impairment.
vii) The compound golexanolone for use according to vi), wherein the motor impairment is selected from any one of, or a combination of, slow movement, tremor, posture, dexterity, gate and balance, communication skills, rigidity, walking and imbalance.
viii) The compound golexanolone for use according to vi), wherein the motor impairment is selected from any one of, or a combination of, slow movement, gate and balance, and walking and imbalance.
ix) The compound golexanolone for use according to any one of i) to v), wherein the Parkinson's Disease (PD) comprises non-motor complications.
x) The compound golexanolone for use according to ix), wherein the non-motor complications are selected from any one of, or a combination of, cognitive impairment, mental health disorders such as depression or anxiety, sleep disorders, pain and sensory disturbances.
xi) The compound golexanolone for use according to ix), wherein the non-motor complications are selected from any one of, or a combination of, cognitive impairment, mental health disorders such as depression or anxiety, and sleep disorders.
xii) The compound golexanolone for use according to vi), wherein the motor impairment is dyskinesia (involuntary movement).
xiii) The compound golexanolone for use according to vi), wherein the motor impairment is dystonia (painful involuntary muscle contractions).
xiv) The compound golexanolone for use according to vi), wherein the motor impairment is slow movement and/or rigidity.
xv) The compound golexanolone for use according to vi), wherein the motor impairment is freezing of gate.
xvi) The compound golexanolone for use according to x) or xi), wherein the sleep disorder is Excessive Daytime Sleepiness (EDS).
xvii) The compound golexanolone for use according to any one of ii), iv) or v), wherein the L-dopa Induced Dyskinesia (LID) comprises hyperkinetic movement.
xviii) The compound golexanolone for use according to xvii) wherein the hyperkinetic movement comprises any one or a combination of chorea, dystonia, or athetosis.
xix) The compound golexanolone for use according to any one of ii), iv) or v), wherein the L-dopa Induced Dyskinesia (LID) is peak-dose dyskinesia (PDD).
xx) The compound golexanolone for use according to any one of ii), iv) or v), wherein the L-dopa Induced Dyskinesia (LID) is diphasic dyskinesia (DD).
xxi) The compound golexanolone for use according to any one of ii), iv) or v), wherein the L-dopa Induced Dyskinesia (LID) is off-period dyskinesia.
xxii) A method for the treatment of Parkinson's Disease (PD), wherein the compound golexanolone (3α-ethynyl-3β hydroxyandrostan-17-one oxime) of formula (I)

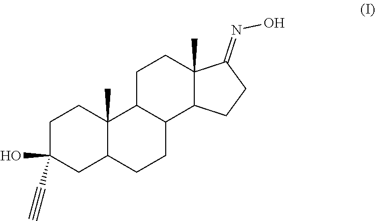 [SMILES: C#C[C@]1(O)CC[C@@]2(C)C(CCC3C2CC[C@]2(C)/C(=N/O)CCC32)C1]

or a pharmaceutically acceptable salt thereof, is administered to a subject in need of such treatment.
xxiii) The method of xxii), wherein the subject exhibits a L-dopa Induced Dyskinesia (LID).
xxiv) Use of the compound golexanolone (3α-ethynyl-3β hydroxyandrostan-17-one oxime) of formula (I)

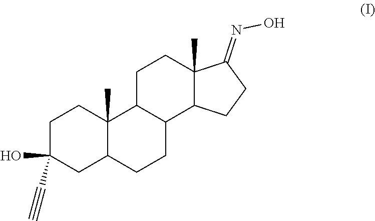 [SMILES: C#C[C@]1(O)CC[C@@]2(C)C(CCC3C2CC[C@]2(C)/C(=N/O)CCC32)C1]

or a pharmaceutically acceptable salt thereof, for the manufacture of a medicament for the treatment of Parkinson's Disease (PD).
xxv) The use of xxiv), wherein the treatment is in PD patients that exhibit a L-dopa Induced Dyskinesia (LID).
xxvi) Use of the compound golexanolone (3α-ethynyl-3β hydroxyandrostan-17-one oxime) of formula (I)

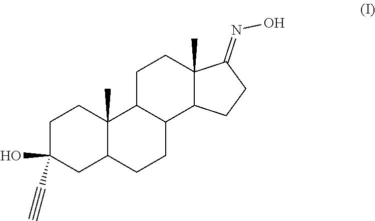 [SMILES: C#C[C@]1(O)CC[C@@]2(C)C(CCC3C2CC[C@]2(C)/C(=N/O)CCC32)C1]

or a pharmaceutically acceptable salt thereof, for the manufacture of a medicament for the treatment of Parkinson's Disease (PD) patients.
xxvii) The use of xxvi), wherein the PD patients exhibit a L-dopa Induced Dyskinesia (LID).

### BRIEF DESCRIPTION OF DRAWINGS

FIG. 1 is a schematic overview of the experimental design used herein, on rats with various symptoms of Parkinson's Disease (PD).

FIG. 2 A and FIG. 2B shows the test results for fatigue.

FIG. 3A shows the test results for motor coordination.

FIG. 3B illustrates the horizontal ladder which rats must cross in the CatWalk test.

FIG. 4A, FIG. 4B, and FIG. 4C, shows the test results for initial dual stance in the CatWalk test.

FIG. 5A and FIG. 5B shows the test results for Locomotor gait swing in the catwalk test.

FIG. 6 shows the test results in the object location memory test (OLM).

FIG. 7 are the test results from short-term spatial memory test.

FIG. 8 shows the test results for an anxiety test.

FIG. 9 shows the test results for a depression test.

FIG. 10 shows the test results of tyrosine hydroxylase (TH) in 6-OHDA rats.

FIG. 11 shows the test results of α-synuclein in 6-OHDA rats.

FIG. 12 shows microglia activation in striatum in 6-OHDA rats.

FIG. 13 shows astrocyte activation in striatum in 6-OHDA rats.

FIG. 14 illustrates the rat experimental set-up for L-dopa Induced Dyskinesia (LID).

FIG. 15 illustrates the evaluation of L-dopa Induced Dyskinesia (LID) AIMs.

FIG. 16 shows all AIMs during three hours from treatment with placebo and golexanolone, respectively.

Throughout the figures, the following abbreviations used, has the following meaning:

SHAM VH or SMVH means sham operated rats treated with vehicle; SHAM GR or SMGR means sham operated rats treated with golexanolone; PARK VH or PKVH means Parkinson rats treated with vehicle; and PARK GR or PKGR means Parkinson rats treated with golexanolone.

### DETAILED DESCRIPTION OF THE INVENTION

An aspect of the present invention is the compound golexanolone (3α-ethynyl-3β hydroxyandrostan-17-one oxime) of formula (I)

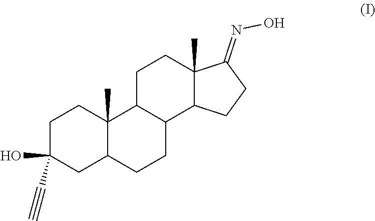 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of Parkinson's Disease (PD).

In another aspect of the present invention, the treatment is in Parkinson's Disease (PD) patients that exhibit a L-dopa Induced Dyskinesia (LID).

A further aspect of the present invention is the compound golexanolone (3α-ethynyl-3β hydroxyandrostan-17-one oxime) of formula (I)

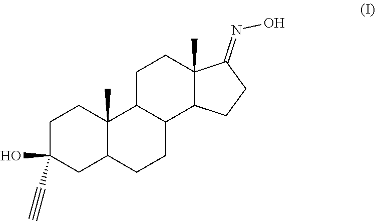 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of Parkinson's Disease (PD) patients.

In another aspect of the present invention, the PD patients to be treated exhibit a L-dopa induced dyskinesia (LID).

A further aspect of the present invention is the compound golexanolone (3α-ethynyl-3β hydroxyandrostan-17-one oxime) of formula (I)

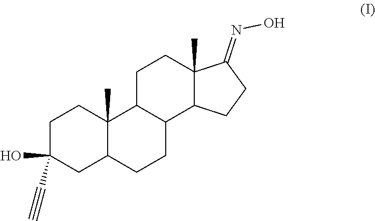 [SMILES: C#C[C@]1(O)CC[C@@]2(C)C(CCC3C2CC[C@]2(C)/C(=N/O)CCC32)C1]

or a pharmaceutically acceptable salt thereof, for use in the treatment of L-dopa induced dyskinesia (LID) in PD patients.

An aspect of the invention is the compound golexanolone for use in the treatment of Parkinson's Disease (PD) comprising motor impairment.

An aspect of the invention is the compound golexanolone for use in the treatment of Parkinson's Disease (PD) comprising motor impairment selected from any one of, or a combination of, slow movement, tremor, posture, dexterity, gate and balance, communication skills, rigidity, freezing of gate, walking and imbalance.

An aspect of the invention is the compound golexanolone for use in the treatment of Parkinson's Disease (PD) comprising non-motor complications.

An aspect of the invention is the compound golexanolone for use in the treatment of Parkinson's Disease (PD) comprising non-motor complications selected from any one of, or a combination of, cognitive impairment, mental health disorders such as depression or anxiety, sleep disorders, pain and sensory disturbances.

An aspect of the invention is the compound golexanolone for use in the treatment of Parkinson's Disease (PD) comprising the motor impairment dyskinesia (involuntary movement).

An aspect of the invention is the compound golexanolone for use in the treatment of Parkinson's Disease (PD) comprising the motor impairment dystonia (painful involuntary muscle contractions).

An aspect of the invention is the compound golexanolone for use in the treatment of Parkinson's Disease (PD) comprising the motor impairment slow movement or rigidity, or a combination of slow movement or rigidity.

An aspect of the invention is the compound golexanolone for use in the treatment of L-dopa Induced Dyskinesia (LID) in PD patients, comprising hyperkinetic movement.

An aspect of the invention is the compound golexanolone for use in the treatment of L-dopa Induced Dyskinesia (LID) in PD patients, comprising hyperkinetic movement selected from chorea, dystonia and athetosis, or a combination thereof.

An aspect of the invention is the compound golexanolone for use in the treatment of L-dopa Induced Dyskinesia (LID) in PD patients, which is peak-dose dyskinesia (PDD).

An aspect of the invention is the compound golexanolone for use in the treatment of L-dopa Induced Dyskinesia (LID) in PD patients, which is diphasic dyskinesia (DD).

An aspect of the invention is the compound golexanolone for use in the treatment of L-dopa Induced Dyskinesia (LID) in PD patients, which is off-period dyskinesia.

An aspect of the present invention is the compound golexanolone for use in preventing or reducing AIMs (Abnormal Involontary Movements) in a subject with symptoms of L-dopa (levodopa) Induced Dyskinesia (LID).

One aspect of the invention is the compound golexanolone for use in Parkinson's Disease comprising a sleep disorder such as Excessive Daytime Sleepiness (EDS).

One aspect of the invention is a method for the treatment of Parkinson's Disease (PD), wherein the compound golexanolone (3α-ethynyl-3β hydroxyandrostan-17-one oxime) of formula (I)

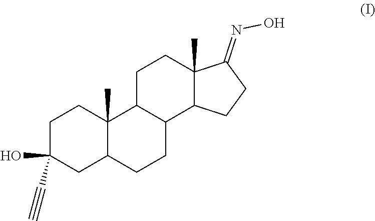 [SMILES: C#C[C@]1(O)CC[C@@]2(C)C(CCC3C2CC[C@]2(C)/C(=N/O)CCC32)C1]

or a pharmaceutically acceptable salt thereof, is administered to a subject in need of such treatment.

In one aspect of the invention, the subject to be treated exhibits a L-dopa Induced Dyskinesia (LID).

One aspect of the invention is the use of the compound golexanolone (3α-ethynyl-3β hydroxyandrostan-17-one oxime) of formula (I)

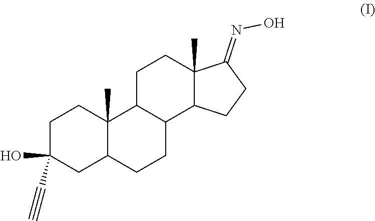 [SMILES: C#C[C@]1(O)CC[C@@]2(C)C(CCC3C2CC[C@]2(C)/C(=N/O)CCC32)C1]

or a pharmaceutically acceptable salt thereof, for the manufacture of a medicament for the treatment of Parkinson's Disease (PD).

In one aspect of the invention, the treatment is in PD patients that exhibit a L-dopa Induced Dyskinesia (LID).

One aspect of the invention is the use of the compound golexanolone (3α-ethynyl-3β hydroxyandrostan-17-one oxime) of formula (I)

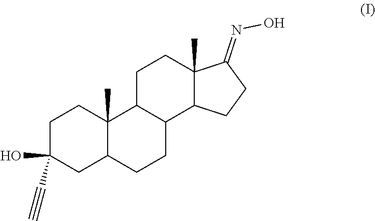 [SMILES: C#C[C@]1(O)CC[C@@]2(C)C(CCC3C2CC[C@]2(C)/C(=N/O)CCC32)C1]

or a pharmaceutically acceptable salt thereof, for the manufacture of a medicament for the treatment of Parkinson's Disease (PD) patients.

In one aspect of the invention, the PD patients exhibit a L-dopa Induced Dyskinesia (LID).

### Definitions

As used throughout the present specification and claims, the compound golexanolone is the compound with the chemical name 3α-ethynyl-3β-hydroxyandrostan-17-one oxime, having the chemical formula (I),

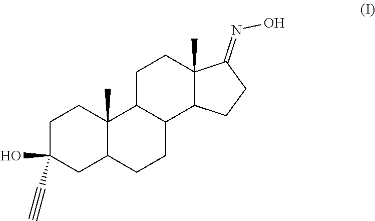 [SMILES: C#C[C@]1(O)CC[C@@]2(C)C(CCC3C2CC[C@]2(C)/C(=N/O)CCC32)C1]

The International Nonproprietary Name (INN) is “golexanolone” and the CAS no. is 2089238-18-4.

Also within the scope of the invention is a pharmaceutically acceptable salt of the compound golexanolone, for use as described and claimed herein.

The compound golexanolone (3α-ethynyl-3β-hydroxyandrostan-17-one oxime) may be prepared according to the method of preparation disclosed in WO 2008/063128.

The wording Parkinson's Disease (PD) is defined herein in accordance with the WHO definition as of 13 Jun. 2022 (WHO 13 Jun. 2022: Parkinson disease (who.int), and means a degenerative condition of the brain associated with motor symptoms (motor impairments) such as slow movement, tremor, rigidity, walking, and imbalance, or any combination thereof.

The wording motor symptoms (motor impairments) means dyskinesias (involuntary movements) and dystonias (painful involuntary muscle contractions). Motor impairment may comprise slow movement, tremor, body posture problems, dexterity problems, gate and balance problems (imbalance), communication (speech) problems, rigidity problems, or walking problems, or any combination thereof.

The wording dyskinesias means involuntary, erratic, writhing movements of the face, arms, legs or trunk, and may also comprise extended muscle spasms, or any combination thereof.

Dyskinesia is a complication from therapy with Parkinson's medications such as levodopa, and usually begin after a few years of treatment with levodopa. This is defined as L-dopa (levodopa) Induced Dyskinesia (LID).

The wording AIMs means Abnormal Involontary Movements which are symptoms of L-dopa (levodopa) Induced Dyskinesia (LID) and comprises axial AIMs, limb AIMs, locomotive AIMs and orolingual AIMs.

The wording Excessive Daytime Sleepiness (EDS) means inability to stay awake and alert during major waking episodes of the day, resulting in periods of irrepressible need for sleep or unintended lapses into drowsiness or sleep.

The wording dystonias means painful involuntary muscle contractions.

Parkinson's Disease (PD) as used herein may further comprise non-motor complications such as fatigue, cognitive impairment, mental health disorders such as depression and anxiety, sleep disorders and pain, as well as other sensory disturbances as described by LeWittAP et al 2020: Parkinsonism and Related Disorders, 80: 57-512.

The wording non-motor complications as used herein is defined in accordance with LeWittAP et al 2020: Parkinsonism and Related Disorders, 80: 57-512, and includes any one of, or a combination of, cognitive impairment, mental health disorders such as depression or anxiety, sleep disorders, pain and sensory disturbances,

The wording L-dopa Induced Dyskinesia (LID) comprises hyperkinetic movement. It may occur during long term duration of treatment with levodopa. LID may also be present in a patient suffering from Parkinson's Disease (PD) after as short as only a few days or months of treatment with levodopa.

L-dopa Induced Dyskinesia (LID) is broadly classified as peak-dose dyskinesia, wearing-off or off-period dyskinesia, and diphasic dyskinesia. Examples of hyperkinetic movement are chorea, dystonia and athetosis, or a combination thereof. The different types of Dyskenesia in L-dopa Induced Dyskinesia (LID) are as defined in Annals of Indian Academy of Neurology; Volume 20; Issue 3; July-September 2017; pp. 190-198 and Fabrini et al: Journal of Experimental Pharmacology 2021:13 pp. 469-485.

The wording peak-dose dyskinesia (PDD) is the most common type of dyskinesia (80%), which occurs at the time of peak plasma levels of levodopa, and is characterized by stereotypic head movements, choreiform truncal movement, and ballistic limb movement, rarely myoclonus (involuntary jerky movements), can be ocular, respiratory, or abdominal muscle.

The wording wearing-off, also referred to as off-period dyskinesia, is the second most common type (30%) of L-dopa Induced Dyskinesia (LID) and typically occurs as early morning dystonia, before the first dose of levodopa, usually involves prolonged spasms and postures in the leg or feet.

The wording diphasic dyskinesia (DD), also referred to as Dyskinesia-improvement-dyskinesia (DID), is the least common (20%) and starts 10-15 min after levodopa ingestion with ipsilateral leg movement and then contralateral involvement, followed by improvement of parkinsonian symptoms for several hours and then recurrence of dyskinesia, when levodopa levels decline. This type of LID occurrs when the levodopa drug concentration in the blood rises or falls.

The wording athetosis means a symptom characterized by slow, involuntary, convoluted, writhing movements of the fingers, hands, toes, and feet and in some cases, arms, legs, neck and tongue.

### Pharmaceutical Formulations and Administration Routes

In certain aspects of the invention, the compound golexanolone (3α-ethynyl-3β-hydroxyandrostan-17-one oxime) as used throughout the present specification and claims, may be administered in the form of a pharmaceutical composition, in admixture with one or more pharmaceutically acceptable adjuvants, diluents and/or carriers. Examples of such pharmaceutically acceptable excipients, carriers and/or diluents useful when formulating the compound golexanolone (3α-ethynyl-3β-hydroxyandrostan-17-one oxime) for use in accordance with the present invention, are thickeners, flavoring agents, diluents, emulsifiers, dispersing aids, carrier substances, lubricants or binders. Typical pharmaceutical carriers include, but are not limited to, binding agents (e.g., pregelatinised maize starch); fillers (e.g., lactose, glucose, sucrose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, and sodium starch glycolate); wetting agents; diluents; coloring agents; emulsifying agents; pH buffering agents; preservatives; and mixtures thereof.

In one aspect of the invention, the compound golexanolone (3α-ethynyl-3β-hydroxyandrostan-17-one oxime) may be administered by enteral administration when used as disclosed and claimed herein. Examples of enteral administration involves administration to the esophagus, stomach, and small and large intestines (i.e. the gastrointestinal tract). Methods of administration include oral, sublingual (dissolving the drug under the tongue), and rectal.

The physician will be able to determine the actual dosage of the compound golexanolone (3α-ethynyl-3β-hydroxyandrostan-17-one oxime) which will be suitable for an individual patient in order to treat Parkinson's Diseases (PD) such as L-dopa induced dyskinesia (LID). The dosage may vary with the route of administration, the severity of the disease, as well as the species, age, weight, and sex, of the patient.

In one aspect of the invention, the compound golexanolone (3α-ethynyl-3β-hydroxyandrostan-17-one oxime) may be administered as a daily dose of from 1 mg to 200 mg, 10 mg to 100 mg, 3 mg to 30 mg, 30 mg to 60 mg, 50 mg to 100 mg, 20 mg to 160 mg, 40 mg to 160 mg, or 80 mg to 160 mg.

The wording “daily dose” may be administration of the compound golexanolone (3α-ethynyl-3β-hydroxyandrostan-17-one oxime) once daily (Q.D.), or twice daily (B.I.D.). Administration twice daily (B.I.D.) means that the total daily dose is divided into two doses which in total makes up the daily dose. For example, a daily dose of 1 mg to 200 mg may be administered as a dose of 1-200 mg once daily (Q.D.), or as a dose of 0.5-100 mg twice daily (B.I.D.).

In one aspect of the invention, a daily dose of the compound golexanolone (3α-ethynyl-3β-hydroxyandrostan-17-one oxime) which may be useful in accordance with the present invention may be selected from any one of 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, and 160 mg.

### EXAMPLES

### Manufacture of the Compound Golexanolone

The compound golexanolone (3α-ethynyl-3β-hydroxyandrostan-17-one oxime) may be prepared according to the method of preparation disclosed in WO 2008/063128.

### Golexanolone Formulation Used in the Experiments

Vehicle or the compound golexanolone (3α-ethynyl-3β-hydroxyandrostan-17-one oxime) was used in the experiments described below.

Golexanolone was administered to the rats as a pharmaceutical formulation which comprises golexanolone formulated in a mixture of mono- and diglycerides of capric/caprylic acid (Capmul® MCM EP/NF; Barentz ApS Odense, Denmark).
(i) Golexanolone is administered in an amount of 50 mg/kg as a solution of 1.25 mL/kg (40 mg/mL of golexanolone in a mixture of mono- and diglycerides of capric/caprylic acid as described above).
(ii) The vehicle (placebo) is a mixture of mono- and diglycerides of capric/caprylic acid as described above without golexanolone, which is administered with a volume of 1.25 mL/kg.

### Biological Evaluation

### Study Design

A 6-OHDA rat model as described by Heo et al. (Curr Biol. 2020 Jan. 20; 30(2): pp. 276-291) and by Carvalho et al. (Mol. Neurodegener. 2013, 8: 14: Behavioral characterization of the 6-hydroxidopamine model of Parkinson's disease and pharmacological rescuing of non-motor deficits) is used.

The model was used to assess:
1) If daily treatment with golexanolone improves motor symptoms (motor coordination, locomotor gait) and non-motor symptoms (cognitive function, fatigue, anxiety, and depression); and
2) If this is associated with improvement of microglia and astrocytes activation, tyrosine hydroxylase (TH) expression and reduced α-synuclein in striatum.

Neuroinflammation in terms of the activation of microglia and astrocytes is a target for reducing the progression of the disease, and α-synuclein and tyrosine hydroxylase (TH) are biomarkers for the pathophysiology linked to neurodegeneration and loss of dopaminergic neurons, respectively, (Carvalho, M. M. 2013. Mol. Neurodegener., 8, 14: Behavioral characterization of the 6-hydroxidopamine model of Parkinson's disease and pharmacological rescuing of non-motor deficits).

Under general anesthesia, all rats receive unilateral injections of 8 μg 6-OHDA (Sigma aldrich) in 4 μL of saline with 0.1% ascorbic acid into the right medial forebrain bundle (AP −2.2 mm, L+1.5 mm relative to the bregma, and V −8.0 mm from the dura) (Paxinos G, Watson C. The Rat Brain in Stereotaxic Coordinates. 4. San Diego, USA: Academic; 1998) with the tooth bar set at +4.5 mm.

To confirm if 6-OHDA model is successfully prepared, the apomorphine (0.25 mg/kg, s.c. administration, Sigma aldrich)-induced rotation test is performed by manual counting. Inclusion criteria is above 6 rpm.

### I. Surgery: Unilateral 6-OHDA Lesion

The unilaterally 6-hydroxydopamine (6-OHDA) rat model according to Carvalho M et al. in Molecular Neurodegeneration 2013, 8:14; pp. 1-11, was used.

Eight week old Wistar-Han male rats (56 animals) (Charles River, Barcelona) were housed, two per cage, under standard laboratory conditions: 12 hour light-dark cycle, 22° C. room temperature, 55% relative humidity, food and water available ad libitum. All manipulations were done in accordance with the local regulations (European Union Directive 2010/63/EU).

Under isoflurane (5% induction and then 2%) anesthesia the animals were placed on a stereotaxic frame with non-traumatic ear bars (Stoelting, USA), and unilaterally injected (left hemisphere) using an 30-gauge needle Hamilton syringe (Hamilton Company, Switzerland), with either vehicle (sham group, n=20) or 6-OHDA hydrochloride (Sigma, USA) (6-OHDA group, n=40) directly into the medial forebrain bundle (coordinates related to Bregma, AP=−4.4 mm; ML=−1.0 mm; DV=−7.8 mm; according to (Paxinos G, Watson C. The Rat Brain in Stereotaxic Coordinates. 4. San Diego, USA: Academic; 1998).

At a rate of 1 μl/min, sham animals received 2 μl of 0.2 mg/ml ascorbic acid in 0.9% NaCl, and 6-OHDA animals were injected with 2 μl 6-OHDA hydrochloride (4 μg/μl) with 0.2 mg/ml ascorbic acid in 0.9% NaCl. After injection the syringe was left in place for 10 minutes to allow diffusion.

### II. Experimental Design to Test the Effect of Golexanolone on Symptoms of Parkinson's Disease (PD)

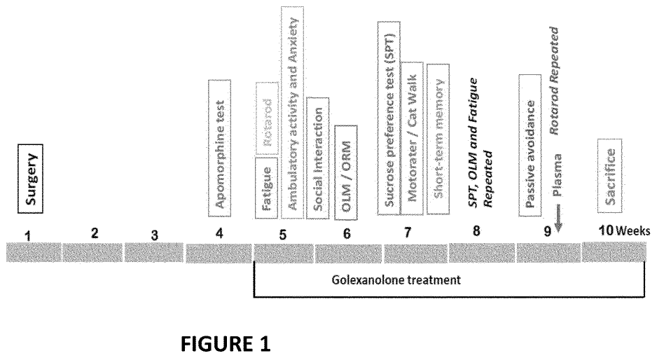

The experimental design is illustrated in FIG. 1.

Rats positive in the apomorphine-induced rotation test (20 animals) were included in the study, together with 20 sham-operated controls. Golexanolone treatment (50 mg/kg, daily, intragastric) and vehicle treatment as negative controls started 4 weeks after surgery. The apomorphine test is a well-known indicator of therapeutic responsiveness to dopaminergic substances and a reliable instrument for the differential diagnosis of i Parkinson's disease. The rats were divided into four groups of sham operated rats treated with vehicle; sham operated rats treated with golexanolone; Parkinson rats treated with vehicle; and Parkinson rats treated with golexanolone, respectively.

The following symptoms in rats with Parkinson's Disease were evaluated:

### Example 1

Fatigue was evaluated in a treadmill consisting of a motorized conveyor belt divided into two parallel sectors, each with an electrified grid. To avoid foot shocks (0.5 mV, 1 mA, 0.3 Hz), the animals have to walk forward without falling off the belt. At room temperature, animals are pre-trained for one day, first exploring for 3 min without starting. Then at 10 cm/s for 5 min and then at 20 cm/s for 5 min. The test is carried out on next day. The animals are then placed on the stationary belt inclined at 52 with a gradually increasing speed of up to 30 cm/s for 5 min and continue at that speed for another 15 min (20 min total). A sensor measures the time spent on the belt. The value recorded during the last 15 minutes of each test (and the times it falls on the grid) is recorded. (Butterworth R F, Lalonde R, Power C, Baker G B, Gamrani H, Ahboucha S. Dehydroepiandrosterone sulphate improves cholestasis-associated fatigue in bile duct ligated rats. Neurogastroenterol Motil. 2009 December; 21(12): pp. 1319-1325).

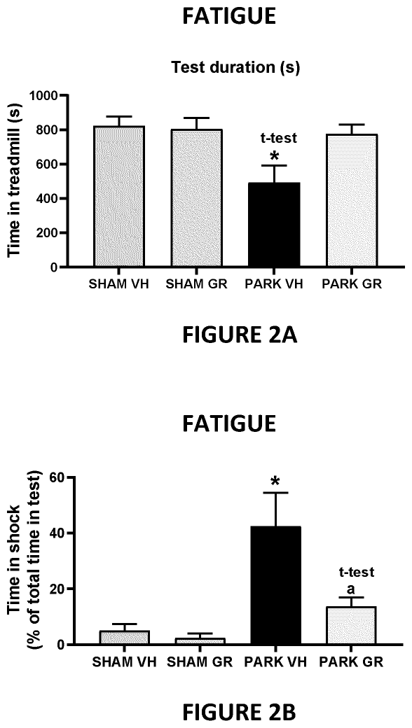

A reduction in the time in the treadmill (A) indicates increased fatigue, which is also reflected in an increase in the time in shock (B). A seen in FIG. 2 A and FIG. 2B, golexanolone improved fatigue after 5 weeks of surgery and 9 days of treatment with golexanolone.

### Example 2

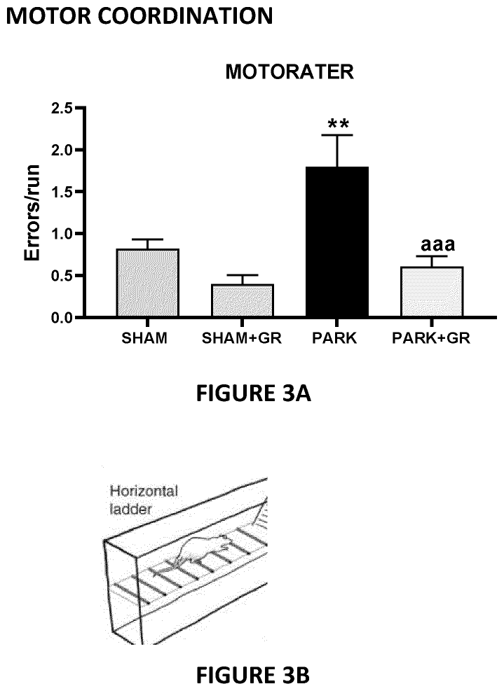

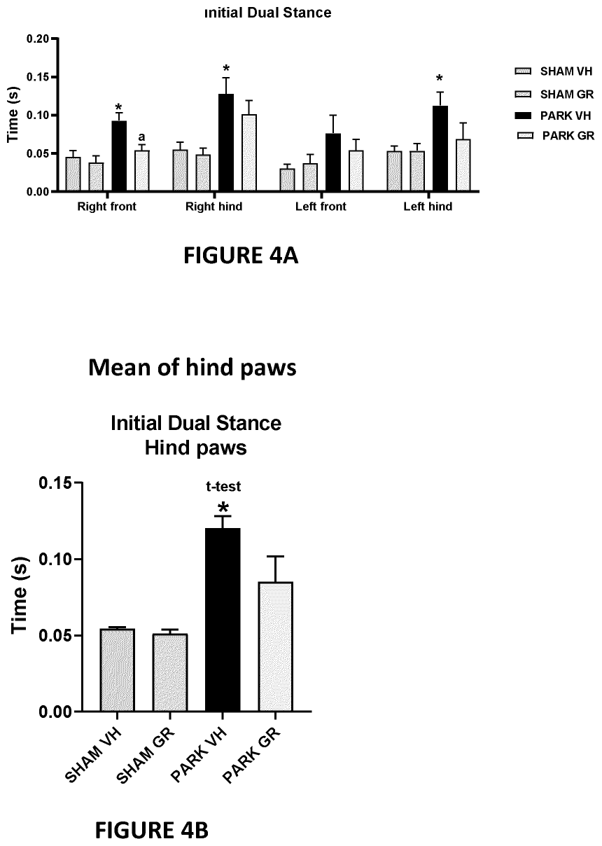

Motor coordination was tested with the Motorater test (Zörner B, Filli L, Starkey M L, Gonzenbach R, Kasper H, Röthlisberger M, et al. Profiling locomotor recovery: comprehensive quantification of impairments after CNS damage in rodents. Nat Methods. 2010; 7(9): pp. 701-708). In this test, the rats must cross a horizontal ladder (FIG. 3B). A kinematic analysis of locomotor performance is then conducted using the MotoRater apparatus (TSE Systems, Germany). In brief, the rats were trained to cross an illuminated glass-walled corridor to reach the dark escape box at the end. 24 hour later rats were tested to cross a ladder. The performance is recorded from the bottom by a mobile high-speed camera at 200 frames per second. Failures to set a foot on the ladder stairs are quantified. FIG. 3A shows the effect after 7 weeks of surgery and 3 weeks of treatment of rats with Parkinson's Disease. As seen in this figure, rats treated with golexanolone, made less errors in making it across the ladder, than the control rats.

### Example 3

Motor coordination was further investigated with the CatWalk system (Lucas E K, Reid C S, McMeekin Li, Dougherty S E, Floyd C L, Cowell R M. Cerebellar transcriptional alterations with Purkinje cell dysfunction and loss in mice lacking PGC-1α. Front Cell Neurosci. 2015; 8:441). (Example 3 and Example 4).

The CatWalk™ system measures various aspects of locomotor pattern. Based on the position, pressure, and surface area of each footfall, multiple parameters are calculated. Trials in which the animal stopped or changed direction are excluded from subsequent analysis. Three uninterrupted trials were performed. Paw print designations were assigned and data analysed using the CatWalk analysis software (v 7.1)

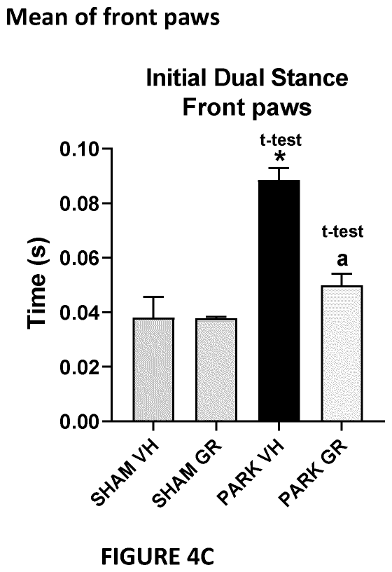

Initial dual stance was evaluated in the catwalk test. Initial dual distance is the duration of contact with the ground of the two hind or front paws at the same time in each step. As seen in FIG. 4A, FIG. 4B, and FIG. 4C, golexanolone improves initial dual stance in the Catwalk test.

### Example 4

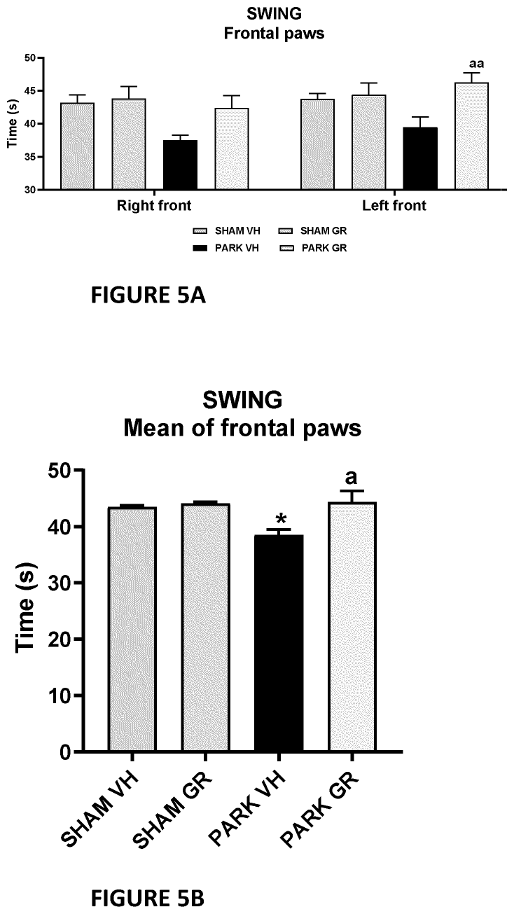

Locomotor gait swing was evaluated in the catwalk test. FIG. 5A and FIG. 5B shows the effect after 7 weeks of surgery and 3 weeks of treatment of rats with Parkinson's Disease. Swing is the time in which one paw is not in contact with the ground. As shown in FIG. 5, rats treated with golexanolone improved swing. The increase in initial dual stance and the decrease in swing indicates that rats with Parkinson's keep their paws raised in each step less time than sham rats. Golexanolone improves this alteration.

### Example 5

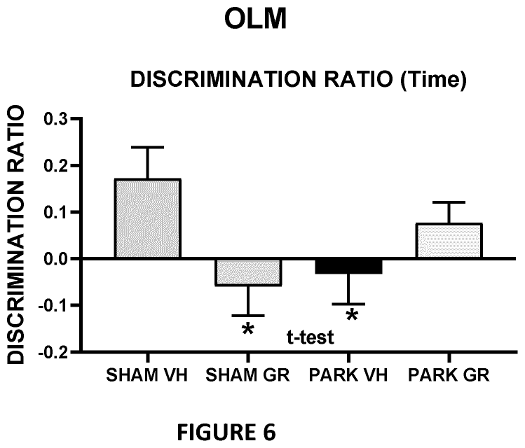

The object location memory test (OLM) evaluates spatial location memory. Rats are put in a field containing two identical objects. Two hours later the location of an object is changed. The time exploring each object is analyzed. The discrimination ratio is calculated: (Time exploring new object location-time exploring old located object)/total time exploring the objects. A lower ratio indicates impaired spatial location memory. As seen from FIG. 6, the results are better in Parkinson's rats treated with golexanolone than in controls receving it, suggesting that golexanolone improves object location memory (OLM). This test was performed after 6 weeks of surgery and 2 weeks of treatment.

### Example 6

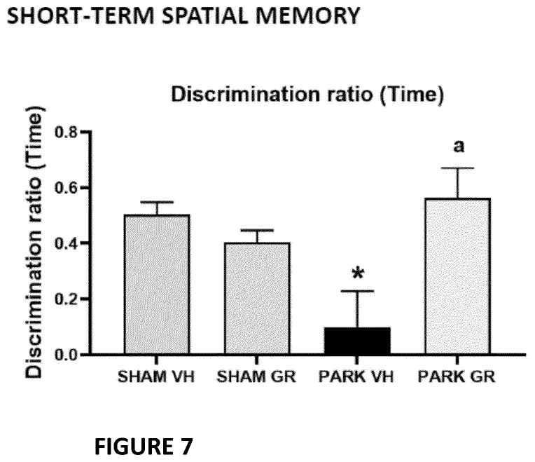

Rats explore a Y-maze with one of the three arms closed. The arm is then opened and after 1 minute the rats are placed back in the maze and should explore the newly opened arm for a longer time. If short-term memory is impaired, the preference for the “new” arm is reduced. We calculate a ratio: (time in the new arm-time in the old arm)/total exploration time. A reduced ratio indicates worse short-term spatial memory. This test was performed after 7 weeks of surgery and 3 weeks of treatment. As seen from FIG. 7.

### Example 7

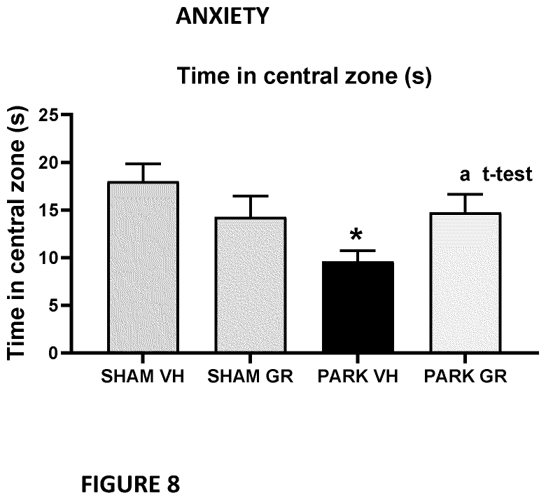

Anxiety was evaluated in an open arena during five minutes by measuring the time that the rat remains in the central zone, instead of close to the walls of the box, where the rats feel safer. Less time in the central zone indicates more anxiety-like behavior. This test was performed after 5 weeks of surgery and 10 days of treatment. As seen in FIG. 8, rats treated with golexanolone improves anxiety.

### Example 8

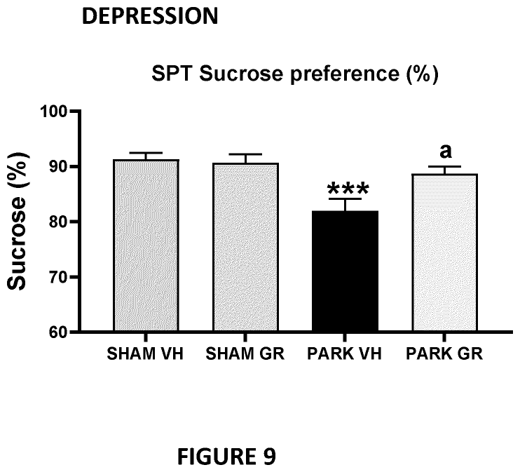

This test was performed after 7 weeks of surgery and 3 weeks of treatment. The sucrose preference test evaluates anhedonia, a symptom of depression. The test consists in measuring the preference for drinking a solution with 1% sucrose compared with water. It is performed after depriving rats of food and drink overnight. The percentage of ml of sucrose solution drunk from the total ml of liquid drunk in two hours, is represented. Reduced preference for the sucrose solution indicates anhedonia (depression) in the rats. As seen in FIG. 9, rats treated with golexanolone improves depression.

### Example 9

### Analysis of Tyrosine Hydroxylase Content

Tyroxine hydroxylase (TH) was analysed according to Heo et al (Curr Biol. 2020 Jan. 20; 30(2): pp. 276-291). Immunohistochemistry staining of TH was performed at 5 weeks after surgery.

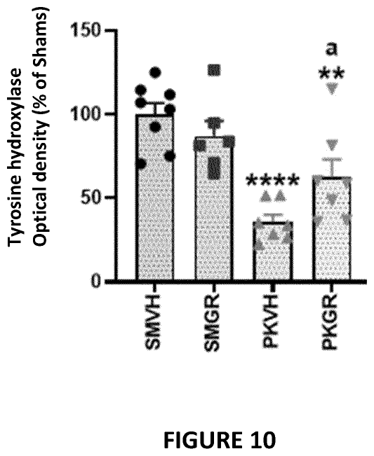

At 5 weeks after surgery, TH staining was reduced in the striatum of 6-OHDA rats (36±4% of shams, p<0.0001). Golexanolone treatment significantly reduced the decrease in TH staining in 6-OHDA rats (62±11% of shams, p<0.05 compared with 6-OHDA untreated rats and p<0.01 compared with shams). The results are shown in FIG. 10.

### Example 10

### Analysis of α-Synuclein Content by Western Blot

α-synuclein content was analyzed by Western blot. Animals were sacrificed by decapitation 10 weeks after surgery. Striata were dissected and homogenized by sonication. Samples were subjected to electrophoresis and immunoblotting as in Felipo et al; Induction of rat brain tubulin following ammonium ingestion. J. Neurochem.; 51(4), pp. 1041-1405, using an α-synuclein antibody (1:1000, Proteintech). Membranes were scanned using the ScanJet 5300C (Hewlett-Packard, Amsterdam, the Netherlands) and band intensities were quantified using Alpha Imager 2200 version 3.1.3 (Alpha Innotech Corporation). Results were expressed as percentage of shams.

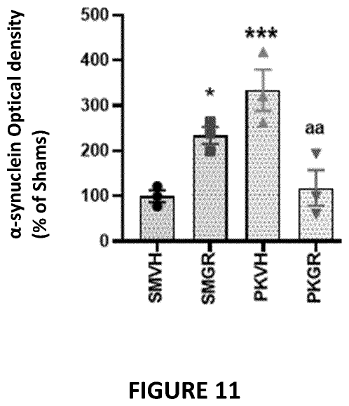

At 10 weeks after surgery, the level of α-synuclein had increased (195±35% of shams, p<0.05) in the injured striatum of 6-OHDA rats. This increase was prevented by golexanolone (99±9% of shams, p<0.05 compared with untreated 6-OHDA rats). The results are shown in FIG. 11.

### Example 11

### Analysis of Microglia Activation

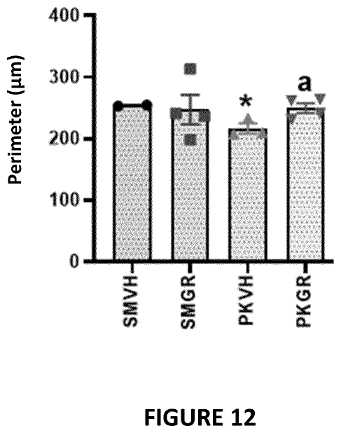

In this Parkinson's Disease test model, microglia activation was analyzed by measuring the perimeter of individual Iba1 stained cells with IpWin2 software at 5 weeks after surgery. Microglia were significantly activated in 6-OHDA rats in the injured striatum at 5 weeks (217±8 μm compared with 254±1 μm in shams, p<0.05). Golexanolone significantly reduced microglia activation in the injured striatum at 5 weeks (250±8 μm, p<0.05 compared with untreated 6-OHDA rats). The results are shown in FIG. 12.

### Example 12

### Analysis of Astrocytes Activation

Astrocytes activation was assessed at 5 weeks after surgery, by measuring the total GFAP stained area with Image J software. Results were expressed as percentage of shams.

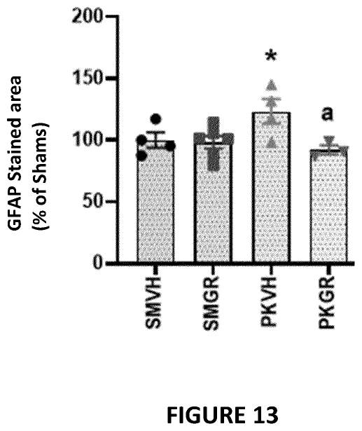

Astrocyte activation in the striatum was analysed by immunohistochemistry. Astrocytes were significantly activated in 6-OHDA rats in the injured striatum (123±10% of shams, p<0.05) and golexanolone reversed this increase (92±4% of shams, p<0.05 compared with untreated 6-OHDA rats). The results are shown on FIG. 13.

### Example 13

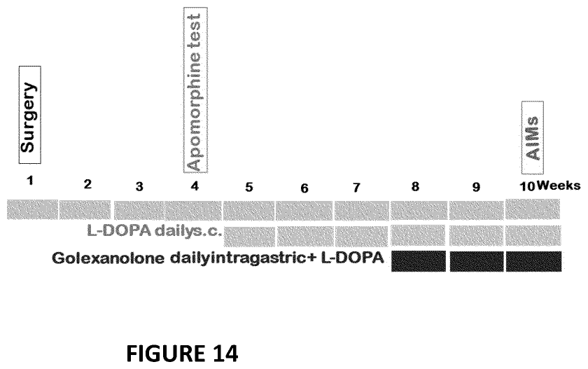

The ability of golexanolone to reduce or eliminate L-dopa Induced Dyskinesia (LID) was analyzed. This test is described in FIG. 14.

L-dopa Induced Dyskinesia (LID) was induced in a rat model described by Loiodice et al in ACS Chem Neurosci. 2018 Apr. 18; 9(4): pp. 762-772. Abnormal Involuntary Movements (AIM) was measured.

Rats positive in the apomorphine test received daily L-DOPA treatment starting 4 weeks after surgery and oral golexanolone treatment starting 7 weeks after surgery. The experimental set-up is shown in FIG. 14.

The experiment included the following groups:
1) 6-OHDA rats treated with L-DOPA and receiving vehicle;
2) 6-OHDA rats treated with golexanolone and treated with L-DOPA (n=12 per group). The appearance of a variety of AIMs were analyzed after 6 weeks of daily L-DOPA injections, and 3 weeks of golexanolone treatment, as described in Lundblad M et al; Eur J Neurosci. 2002 January; 15(1): pp. 120-132, and Loiodice et al in ACS Chem Neurosci. 2018 Apr. 18; 9(4): pp. 762-772.

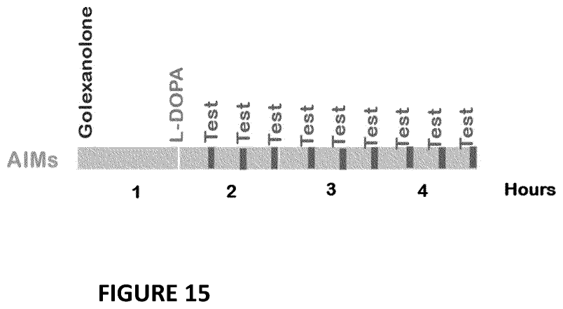

Axial AIMs, limb AIMs, orolingual AIMs and locomotive AIMs were evaluated. One hour after the daily dose of golexanolone, L-DOPA was injected and AIMs were analyzed every 20 minutes for a total of three hours, as shown in FIG. 15.

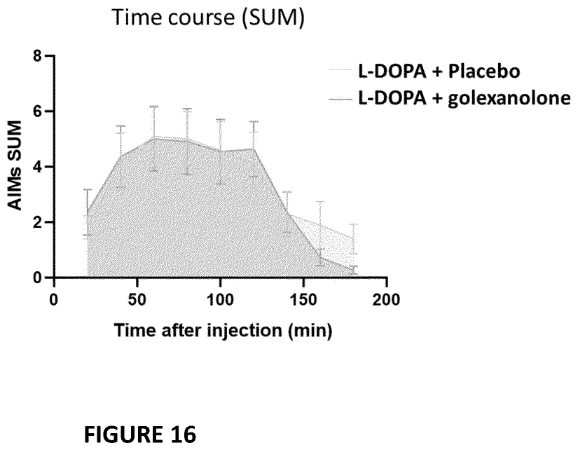

FIG. 16 shows the time course of all AIMs (axial AIMs, limb AIMs, orolingual AIMs and locomotive AIMs) during the total three hours. As shown in this graph, there were less AIMs in the group of animals treated with golexanolone compared to the group who received only L-DOPA.

### Statistical Analysis

Data are expressed as mean±SEM. All statistical analyses were performed using GraphPad Prism software v. 9.0. Data were tested for normality (Kolmogorov-Smirnov or Shapiro-Wilk test) and for homogeneity of variances. Statistical analysis was carried out using one-way ANOVA and Tukey's or Fisher's multiple comparisons test or two-way ANOVA when appropriate. When data did not pass the normality test, the nonparametric Kruskal-Wallis test, with Dunn's test for multiple comparisons, was used. When standard deviations (SDs) were not equal, Welch's ANOVA was used.

1. A method for the treatment of Parkinson's Disease (PD), the method comprising administering the compound golexanolone (3α-ethynyl-3β hydroxyandrostan-17-one oxime) of formula (I) 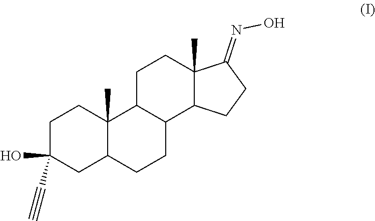 [SMILES: C#C[C@]1(O)CC[C@@]2(C)C(CCC3C2CC[C@]2(C)/C(=N/O)CCC32)C1]
or a pharmaceutically acceptable salt thereof, to a Parkinson's Disease patient in need of such treatment.

2. The method according to claim 1, wherein the Parkinson's Disease (PD) patients exhibits an L-dopa Induced Dyskinesia (LID).

3. (canceled)

4. (canceled)

5. A method for the treatment of L-dopa Induced Dyskinesia (LID) in a Parkinson's Disease (PD) patient, the method comprising administering the compound golexanolone (3α-ethynyl-3β hydroxyandrostan-17-one oxime) of formula (I) 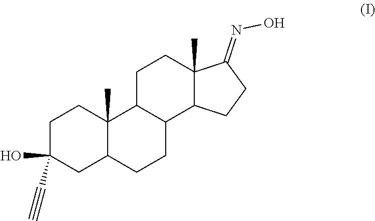 [SMILES: C#C[C@]1(O)CC[C@@]2(C)C(CCC3C2CC[C@]2(C)/C(=N/O)CCC32)C1]
or a pharmaceutically acceptable salt thereof, to the Parkinson's Disease patient in need of such treatment.

6. The method according claim 1, wherein the Parkinson's Disease (PD) comprises motor impairment.

7. The method according to claim 6, wherein the motor impairment is selected from any one of, or a combination of, slow movement, tremor, posture, dexterity, gate and balance, communication skills, rigidity, walking and imbalance.

8. The method according to claim 6, wherein the motor impairment is selected from any one of, or a combination of, slow movement, gate and balance, and walking and imbalance.

9. The method according to claim 1, wherein the Parkinson's Disease (PD) comprises non-motor complications.

10. The method according to claim 9, wherein the non-motor complications are selected from any one of, or a combination of, cognitive impairment, mental health disorders such as depression or anxiety, sleep disorders, pain and sensory disturbances.

11. The method according to claim 9, wherein the non-motor complications are selected from any one of, or a combination of, cognitive impairment, mental health disorders such as depression or anxiety, and sleep disorders.

12. The method according to claim 6, wherein the motor impairment is dyskinesia (involuntary movement).

13. The method according to claim 6, wherein the motor impairment is dystonia (painful involuntary muscle contractions).

14. The method according to claim 6, wherein the motor impairment is slow movement and/or rigidity.

15. The method according to claim 6, wherein the motor impairment is freezing of gate.

16. The method according to claim 10, wherein the sleep disorder is Excessive Daytime Sleepiness (EDS).

17. The method according to claim 2, wherein the L-dopa Induced Dyskinesia (LID) comprises hyperkinetic movement.

18. The method according to claim 17, wherein the hyperkinetic movement comprises any one or a combination of chorea, dystonia, or athetosis.

19. The method according to claim 2, wherein the L-dopa Induced Dyskinesia (LID) is peak-dose dyskinesia (PDD).

20. The method according to claim 2, wherein the L-dopa Induced Dyskinesia (LID) is diphasic dyskinesia (DD).

21. The method according to claim 2, wherein the L-dopa Induced Dyskinesia (LID) is off-period dyskinesia.
